(19) United States
(12) Plant Patent Application Publication
(10) Pub. No.: US 2017/0013761 P1
(43) Pub. Date: Jan. 12, 2017

(54) Chrysanthemum plant named 'MEFISTO DARK RED'

(50) ;

(71) Applicant: Dirk Pieters, Oostnieuwkerke (BE)

(72) Inventor: Dirk Pieters, Oostnieuwkerke (BE)

(73) Assignee: PARATY B.V.B.A., Oostnieuwkerke (BE)

(21) Appl. No.: 14/545,962

(22) Filed: Jul. 10, 2015

(51) Int. Cl. A01H 5/00

(52) U.S. Cl. PLT/298

(57) ABSTRACT

A new and distinct cultivar of Chrysanthemum plant named ‘Mefisto Dark Red’, characterized by its upright, outwardly spreading and uniformly rounded plant habit; moderately vigorous growth habit; medium green-colored leaves; uniform and freely flowering habit; and relatively large decorative-type inflorescences that are dark red in color.

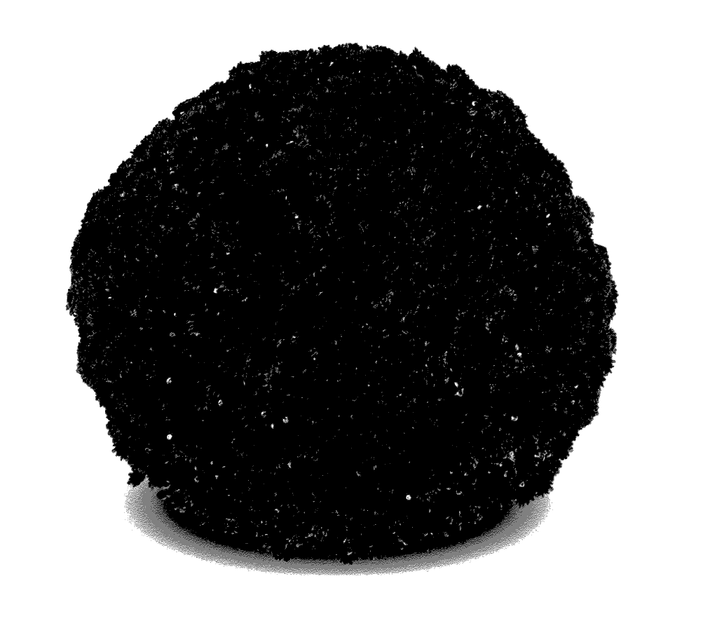

### BOTANICAL DESIGNATION

Chrysanthemum×morifolium

### CULTIVAR DENOMINATION

‘MEFISTO DARK RED’

### BACKGROUND OF THE INVENTION

The present invention relates to a new and distinct cultivar of Chrysanthemum plant, botanically known as Chrysanthemum×morifolium and hereinafter referred to by the name ‘Mefisto Dark Red’. The new Chrysanthemum plant is a product of a planned breeding program conducted by the Inventor in Oostnieuwkerke, Belgium. The objective of the breeding program is to create new uniformly mounding and freely flowering Chrysanthemum plants with unique and attractive ray floret coloration.

The new Chrysanthemum plant is a naturally-occurring whole plant mutation of Chrysanthemum×morifolium ‘Mefisto Red Imp’, not patented. The new Chrysanthemum plant was discovered and selected by the Inventor as a single flowering plant from within a population of plants of ‘Mefisto Red Imp’ in a controlled greenhouse environment in Oostnieuwkerke, Belgium in October, 2012.

Asexual reproduction of the new Chrysanthemum plant by vegetative terminal cuttings was first conducted in a controlled greenhouse environment in Oostnieuwkerke, Belgium in January, 2013. Asexual reproduction by vegetative terminal cuttings has shown that the unique features of this new Chrysanthemum plant are stable and reproduced true to type in successive generations.

### SUMMARY OF THE INVENTION

Plants of the new Chrysanthemum have not been observed under all possible combinations of environmental conditions and cultural practices. The phenotype may vary somewhat with variations in environmental conditions such as temperature, daylength and light intensity, without, however, any variance in genotype.

The following traits have been repeatedly observed and are determined to be the unique characteristics of ‘Mefisto Dark Red’. These characteristics in combination distinguish ‘Mefisto Dark Red’ as a new and distinct Chrysanthemum plant:

1. Upright, outwardly spreading and uniformly rounded plant habit.

2. Moderately vigorous growth habit.

3 Medium green-colored leaves.

4. Uniform and freely flowering habit.

5. Relatively large decorative-type inflorescences that are dark red in color.

Plants of the new Chrysanthemum differ primarily from the parent, ‘Mefisto Red Imp’, in the following characteristics:

1. Plants of the new Chrysanthemum are more compact than plants of ‘Mefisto Red Imp’.

2. Plants of the new Chrysanthemum and ‘Mefisto Red Imp’ differ in ray floret color as plants of the new Chrysanthemum have darker red-colored ray florets than plants of ‘Mefisto Red Imp’. Additionally, ray floret color of plants of the new Chrysanthemum does not fade as quickly as ray floret color of plants of ‘Mefisto Red Imp’.

Plants of the new Chrysanthemum can be compared to plants of Chrysanthemum×morifolium ‘PPP CONT 09’, disclosed in U.S. Plant Pat. No. 21,483. In side-by-side comparisons conducted in Oostnieuwkerke, Belgium, plants of the new Chrysanthemum differed from plants of ‘PPP CONT 09’ in the following characteristics:

1. Plants of the new Chrysanthemum were larger than plants of ‘PPP CONT 09’.

2. Leaves of plants of the new Chrysanthemum were smaller and brighter green in color than leaves of plants of ‘PPP CONT 09’.

3. Plants of the new Chrysanthemum and ‘PPP CONT 09’ differed in ray floret color as plants of ‘PPP CONT 09’ had deeper dark red-colored ray florets.

### BRIEF DESCRIPTION OF THE PHOTOGRAPH

The accompanying photograph illustrates the overall appearance of the new Chrysanthemum plant showing the colors as true as it is reasonably possible to obtain in colored reproductions of this type. Colors in the photograph may differ slightly from the color values cited in the detailed botanical description which accurately describe the colors of the new Chrysanthemum plant. The photograph comprises a side perspective view of a typical flowering plant of ‘Mefisto Dark Red’ grown in a container.

### DETAILED BOTANICAL DESCRIPTION

The aforementioned photograph and following observations and measurements describe plants grown in 19-cm containers in an outdoor nursery in Oostnieuwkerke, Belgium during the spring and summer and under cultural practices generally used in commercial Chrysanthemum production. During the production of the plants, day temperatures ranged from 18° C. to 21° C. and night temperatures ranged from 10° C. to 15° C. Plants were 20 weeks old when the photograph and description were taken. In the following description, color references are made to The Royal Horticultural Society Colour Chart, 2005 Edition, except where general terms of ordinary dictionary significance are used.
Botanical classification: Chrysanthemum×morifolium ‘Mefisto Dark Red’.
Parentage: Naturally-occurring whole plant mutation of Chrysanthemum×morifolium ‘Mefisto Red Imp’, not patented.
Propagation:
   Type.—Vegetative terminal cuttings.
   Time to initiate roots, summer.—About 14 days at temperatures about 20° C.
   Time to initiate roots, winter.—About 20 days at temperatures about 20° C.
   Time to produce a rooted young plant, summer.—About 30 days at temperatures about 20° C.
   Time to produce a rooted young plant, winter.—About 40 days at temperatures about 20° C.
   Root description.—Fine, fibrous; typically light brown in color, actual color of the roots dependent on substrate composition, water quality, fertilizers, substrate temperature and age of roots.
   Rooting habit.—Freely branching; medium density.
Plant description:
   Appearance.—Perennial decorative-type Chrysanthemum; stems upright and outwardly spreading giving a uniformly rounded appearance to the plant; plants nearly spherical in overall shape; very freely branching habit, about 25 primary lateral branches develop, each primary lateral branch with multiple secondary branches; pinching enhances lateral branch development; dense and full plant habit; moderately vigorous growth habit.
   Plant height.—About 40 cm.
   Plant width.—About 60 cm.
   Lateral branches.—Length: About 25 cm. Diameter: About 2 mm to 3 mm. Internode length: About 2 cm. Strength: Strong, flexible. Aspect: Upright to outwardly spreading. Texture: Pubescent; longitudinally ridged. Luster: Matte. Color: Close to 144A.
   Leaves.—Arrangement: Alternate, simple. Length: About 2.5 cm to 5 cm. Width: About 1.5 cm to 3 cm. Apex: Rounded to cuspidate. Base: Attenuate. Margin: Palmately lobed and serrate, sinuses between lateral lobes divergent to parallel. Texture, upper and lower surfaces: Slightly pubescent. Luster, upper and lower surfaces: Matte. Color: Developing leaves, upper surface: Close to 139C. Developing leaves, lower surface: Close to 137D. Fully expanded leaves, upper surface: Close to N137C; venation, close to 148C. Fully expanded leaves, lower surface: Close to 147B; venation, close to 147B to 147C. Petioles: Length: About 1 cm. Diameter: About 2 mm. Texture, upper and lower surfaces: Slightly pubescent and slightly rough. Luster, upper and lower surfaces: Matte. Color, upper surface: Close to 146C. Color, lower surface: Close to 146D.
Inflorescence description:
   Appearance.—Decorative-type inflorescence form; inflorescences borne on terminals above foliar plane; disc and ray florets arranged acropetally on a capitulum.
   Fragrance.—Faintly fragrant, pungent.
   Flowering time.—Under natural season conditions, plants flower in mid-October in Belgium.
   Postproduction longevity.—Inflorescences maintain good color and substance for about five weeks on the plant grown in an outdoor nursery; inflorescences persistent.
   Quantity of inflorescences.—Freely flowering habit with about 20 inflorescences developing per lateral branch with numerous inflorescences developing per plant.
   Inflorescence buds.—Height: About 4 mm. Diameter: About 7 mm. Shape: Globular. Texture: Smooth. Color: Close to 144A and 137C.
   Inflorescence size and shape.—Diameter: Relatively large, about 4.5 cm. Depth (height): About 2.5 cm. Disc diameter: About 1 mm. Shape: Circular; raised dome. Receptacle diameter: About 3 mm. Receptacle height: About 2.5 mm to 3 mm. Receptacle texture: Smooth, glabrous. Receptacle color: Close to 144B.
   Ray florets.—Quantity and arrangement: About 200 or more ray florets per inflorescence arranged in about ten whorls. Length: About 1.3 cm to 2 cm. Width: About 7 mm. Shape: Oval. Apex: Rounded. Base: Attenuate. Margin: Entire. Aspect: Mostly flat. Texture, upper and lower surfaces: Smooth, glabrous. Luster, upper and lower surfaces: Matte. Color: When opening and fully opened, upper surface: Close to 185B; color becoming closer to 184D with development. When opening and fully opened, lower surface: Close to 181 A; color becoming closer to 184D with development.
   Disc florets.—Quantity and arrangement: About 20 or less disc florets per inflorescence and massed at the center of the receptacle; disc florets inconspicuous. Length: About 3 mm. Diameter: About 0.5 mm to 1 mm. Shape: Tubular, elongated; apices acute. Color, immature: Apex: Close to 144C. Mid-section: Close to 5B. Base: Close to 145D. Color, mature: Apex: Close to 12A. Mid-section: Close to 12A. Base: Close to 12A.
   Phyllaries.—Quantity and arrangement: About 25 phyllaries per inflorescence arranged in about two or three whorls. Length: About 4 mm to 6 mm. Width: About 2 mm to 3 mm. Shape: Ovate. Apex: Rounded. Base: Rounded to truncate. Margin: Entire. Texture, upper and lower surfaces: Smooth, glabrous. Luster, upper and lower surfaces: Matte. Color, upper surface: Close to 137A. Color, lower surface: Close to N137B.
   Peduncles.—Length, terminal peduncle: About 4 cm. Length, fourth peduncle: About 6 cm. Length, seventh peduncle: About 8 cm. Diameter: About 2 mm. Angle: About 30° from vertical. Strength: Moderately strong. Texture: Slightly pubescent. Luster: Matte. Color: Close to 146B.
   Reproductive organs.—Androecium: None observed. Gynoecium: None observed.
   Seeds and fruit.—Seed and fruit production have not been observed on plants of the new Chrysanthemum.
Disease & pest resistance: Resistance to pathogens and pests common to Chrysanthemum plants has not been observed on plants of the new Chrysanthemum grown under commercial production conditions.
Garden performance: Plants of the new Chrysanthemum have demonstrated excellent garden performance and will tolerate temperatures ranging from about 0° C. to about 45° C.

It is claimed:

1. A new and distinct Chrysanthemum plant named ‘Mefisto Dark Red’ as illustrated and described.
